(19) United States
(12) Patent Application Publication
(10) Pub. No.: US 2025/0057738 A1
(43) Pub. Date: Feb. 20, 2025

(54) COSMETIC SWEAT MANAGEMENT COMPOSITIONS

(71) Applicant: Conopco, Inc., d/b/a UNILEVER, Englewood Cliffs, NJ (US)

(72) Inventors: Martin Peter CROPPER, Birkenhead (GB); Neil Robert FLETCHER, Irby, Wirral (GB); Veronique Sandrine MUNIER, Liverpool (GB); Joanne Elizabeth STOCKTON, Bebington, Wirral (GB)

(73) Assignee: Conopco, Inc., d/b/a UNILEVER, Englewood Cliffs, NJ (US)

(21) Appl. No.: 18/719,387

(22) PCT Filed: Dec. 2, 2022
(86) PCT No.: PCT/EP2022/084191

§ 371 Date: Jun. 13, 2024

(30) Foreign Application Priority Data
Dec. 22, 2021 (EP) 21217085.6

(51) Int. Cl. A61K 8/04; A61Q 15/00

(52) U.S. Cl. CPC A61K 8/046; A61Q 15/00; A61K 2800/22; A61K 2800/34; A61K 2800/524; A61K 2800/5922; A61K 2800/805

(57) ABSTRACT

An antiperspirant aerosol composition comprising a three-component mixture of propellant, ethanol and a lipid mixture consisting of glycerol monolaurate and isostearyl alcohol at a ratio of from 25:75 to 45:55, wherein the propellant content of the three-component mixture is from 40 to 87.5%; the ethanol content of the three-component mixture is from 10 to 57.5% and the lipid content of the three-component mixture is from 2.5% up to a maximum equal to the sum of 4.805 and the ethanol content multiplied by 0.115.

### FIELD OF INVENTION

The present invention is in the field of cosmetic compositions and their use in sweat management, in particular, non-aluminium cosmetic compositions for such use.

### BACKGROUND

EP 550,960 A1 (Unilever, 1992) discloses the use as an antiperspirant active of a lipid mixture which forms, upon contact with perspiration, a water-insoluble liquid crystal phase of greater than one dimensional periodicity. This publication does not disclose ethanolic compositions, nor their stability issues, nor their use in aerosol compositions.

WO 94/024993 (Unilever, 1994) discloses an antiperspirant composition comprising a lipid mixture which forms, upon contact with perspiration, a water-insoluble liquid crystal phase of greater than one dimensional periodicity, in a cosmetic vehicle comprising a volatile silicone and containing less than 10% by weight of the total composition of a short chain monohydric alcohol. This publication does not disclose compositions with the specific ratios of components as defined herein and does not disclose their importance in the low temperature (0° C.) stability thereof.

WO2020/108882 A1 (Unilever, 2020) discloses antiperspirant aerosol compositions comprising ethanol, amphiphilic material similar to that disclosed in the above publications and volatile silicone, the ratios and amounts of the components being selected to provide good storage stability for the aerosols, particularly at low temperatures.

WO2020/108885 A1 (Unilever, 2020) discloses antiperspirant compositions comprising ethanol, isostearyl alcohol, glycerol monolaurate, volatile silicone and humectant, the ratios and amounts of the components being selected to provide good storage stability.

EP 3,773,433 B1 (Unilever, 2021) discloses antiperspirant compositions comprising ethanol, isostearyl alcohol, glycerol monolaurate and volatile silicone, the ratios of the components being selected to provide good storage stability, particularly at low temperatures.

### SUMMARY OF INVENTION

It is an object of the present invention to provide a cosmetic aerosol composition that does not require the presence of an aluminium salt to deliver sweat management benefits. It is a further of the present invention to do this from a composition that has a high degree of storage stability, particularly at low temperatures.

It is an object of the present invention to provide a cosmetic aerosol composition that is free from silicone oil and which has good stability, particularly at low temperature. It is a further object of the present invention to provide such an aerosol composition that does not require the presence of an aluminium salt to deliver a sweat management benefit.

In a first aspect of the invention, there is provided a cosmetic aerosol composition comprising a three-component mixture of propellant, ethanol and a lipid mixture consisting of glycerol monolaurate and isostearyl alcohol at a ratio of from 25:75 to 45:55 by weight, wherein:
(i) the propellant content of the three-component mixture is from 40 to 87.5% by weight;
(ii) the ethanol content of the three-component mixture is from 10 to 57.5% by weight and
(iii) the lipid content, by weight, of the three-component mixture is from 2.5% up to a maximum equal to the sum of 4.805 and the ethanol content multiplied by 0.115.

In a second aspect of the invention, there is provided a method of manufacture of a cosmetic aerosol composition according to the first aspect of the invention.

In a third aspect of the invention, there is provided a cosmetic method of attaining a sweat management protection benefit comprising the topical application of a composition according to the first aspect of the invention.

### DETAILED DESCRIPTION

Herein, features expressed as “preferred” with regard to a particular aspect of the invention should be understood to be preferred with regard to each aspect of the invention (likewise, features expressed as “more preferred” or “most preferred”).

Herein, preferred features of the invention are particularly preferred when used in combination with other preferred features.

Herein, “ambient conditions” refers to 25° C. and 1 atmosphere pressure, unless otherwise indicated.

Herein, the term “volatile” refers to a material having a boiling point of less 10° C.

Herein, all percentages, ratios and amounts are by weight, unless otherwise indicated.

Herein, the word “comprising” is intended to mean “including” but not necessarily “consisting of”, i.e., it is non-exhaustive.

Herein, “cosmetic” methods and compositions should be understood to mean non-therapeutic methods and compositions, respectively.

Herein, “water-insoluble” means having a solubility in water of less than 0.1% by weight (at 37° C.).

The compositions of the invention are particularly effectively when applied to the underarm regions of the human body and/or the feet. The compositions are especially effectively when applied to the underarm regions of the human body.

Aerosol compositions consist of a propellant and a base. The components of the base are typically mixed together first and the propellant is added last in a process sometimes called “gassing”.

Herein, the “base” of an aerosol composition is all the components of the total composition other than the propellant.

It is important that the fully formulated aerosol composition has good storage stability, so that it can survive prolonged transit to stores and extended periods on shelf prior to purchase and use.

The present invention involves compositions having superior storage stability, particularly at low temperatures, such as 0° C. By achieving stability at low temperatures, the present invention enables effective transit and storage of the claimed compositions in cold regions of the world. In addition, it enables transit and storage at refrigerated temperatures as may be required for the preservation of heat sensitive ingredients of the composition, such as certain fragrance components.

The lipid mixture is a mixture of isostearyl alcohol and glycerol monolaurate at a weight ratio of from 25:75 to 45:55. This lipid mixture is able to form an inverse hexagonal phase following contact with sweat on the surface of the human body and this leads to the antiperspirancy benefit of the compositions of the invention (as disclosed in EP 550,960 A1 [Unilever, 1992]).

The lipid content, by weight, of the tri-component mixture consisting of lipid, propellant and ethanol, is from 2.5% up to a maximum equal to the sum of 4.805 and the ethanol content multiplied by 0.115. The equation defining the maximum ethanol content is derived from the results of multiple stability tests. Together with the limits set for ethanol and propellant levels, the lipid content defines the 3-component claim space for compositions according to the invention.

The content of lipid mixture in the total composition, ignoring the propellant therein, is preferably at least 10%, more preferably at least 12% and most preferably at least 15%. The content of lipid mixture, again ignoring and propellant in the composition, is preferably 45% or less and more preferably 40% or less, these upper amounts being combinable with each of the lower ranges indicated in this paragraph to give preferred ranges of incorporation.

The purpose of the ethanol is principally to solubilise the lipid mixture. The ethanol is present at from 10 to 57.5% of the tri-component mixture consisting of ethanol, lipid mixture and propellant.

The content of ethanol in the total composition, ignoring the propellant therein, is preferably at least 10%, more preferably at least 15% and most preferably at least 30%. The content of ethanol, again ignoring and propellant in the composition, is preferably 95% or less and more preferably 90% or less, these upper amounts being combinable with each of the lower ranges indicated in this paragraph to give preferred ranges of incorporation.

The propellant is commonly either a compressed gas or a material that boils at below ambient temperature, preferably at below 0° C., and especially at below −10° C. Examples of compressed gasses include compressed air, nitrogen and carbon dioxide. Examples of suitable propellants include volatile hydrocarbons, dimethyl ether and hydrofluorocarbons containing from 2 to 4 carbons, at least one hydrogen and 3 to 7 fluorine atoms. In particularly preferred embodiments, the propellant used comprises or is solely a hydrocarbon propellant.

Preferred hydrocarbons for use as propellant include propane, butane, isobutane and mixtures thereof.

When compositions according to the invention are aerosol compositions, they can be made in a conventional manner by first preparing a base composition, charging the base composition into the aerosol can, fitting a valve assembly into the mouth of the can, thereby sealing the can, and thereafter charging propellant into the can to a desired pressure, and finally fitting an actuator on or over the valve assembly.

The propellant content of the three-component mixture is from 40 to 87.5% and preferably from 50 to 85%.

Cosmetic compositions according to the present invention are preferably free from aluminium or zirconium antiperspirant salts.

A preferred additional component for use in compositions of the present invention is a fragrance or fragrance oil, sometimes alternatively called a perfume (oil). The fragrance oil may comprise a single fragrance or component more commonly a plurality of fragrance components. Herein, fragrance oils impart an odour, preferably a pleasant odour, to the composition. Preferably, the fragrance oil imparts a pleasant odour to the surface of the human body the composition is applied to the same.

The amount of fragrance oil in the composition is commonly up to 3% advantageously is at least 0.5% and particularly from 0.8% to 2%.

A preferred additional component of compositions of the invention is a deodorant active. These are typically antimicrobial agents active against bacterial on the skin of the human body. These serve to reduce malodour and especially useful in compositions in which the lipid mixture is not itself an antimicrobial agent. When employed, the level of incorporation is preferably 0.01%-5%, more preferably from 0.01-2% and most preferably from 0.03%-0.5% by weight of the total composition.

Preferred anti-microbial deodorant agents are those that are more efficacious than simple alcohols such as ethanol. Particularly preferred anti-microbial deodorant agents are soluble in ethanol, meaning that they a solubility in ethanol of at least 10 g/L at 20° C. Examples of suitable anti-microbial deodorant agents include niacinamide; quaternary ammonium compounds, like cetyltrimethylammonium salts; chlorhexidine and salts thereof; and diglycerol monocaprate, diglycerol monolaurate, glycerol monolaurate, and similar materials, as described in “Deodorant Ingredients”, S. A. Makin and M. R. Lowry, in “Antiperspirants and Deodorants”, Ed. K. Laden (1999, Marcel Dekker, New York). More preferred are polyhexamethylene biguanide salts (also known as polyaminopropyl biguanide salts), an example being Cosmocil CQ available from Arch Chemicals, 2′,4,4′-trichloro,2-hydroxy-diphenyl ether (triclosan), 3,7,11-trimethyldodeca-2,6,10-trienol (farnesol), essential oils such as Tea Tree Oil and Thyme Oil, climbazole, octapyrox, ketoconazole, zinc pyrithione and mixtures thereof.

A preferred optional component is a preservative, such as ethyl or methyl parabens or BHT (butyl hydroxy toluene), typically in an amount of from 0.01 to 0.1% by weight of the total composition.

The invention will now be described by way of some examples, which do not limit the extent of the invention.

### EXAMPLES

Numerous examples and comparative examples have been prepared and tested. Without exception, the Examples (indicated by numbers) according to the invention had superior stability to the Comparative Examples (indicated by C . . . number).

The compositions detailed in Table 1 were prepared by methods known in the art, the liquid components being mixed first, followed by addition of the propellant. The amounts indicated are parts by weight.

TABLE 1

| Example | Propellant | Ethanol + fragrance | Lipid mixture | Stability |
|---|---|---|---|---|
| 1 | 50.00 | 41.70 | 8.30 | Good |
| C1 | 70.00 | 21.70 | 8.30 | Inferior |
| C2 | 80.00 | 11.70 | 8.30 | Poor |
| C3 | 85.00 | 6.70 | 8.30 | Poor |
| 2 | 60.00 | 33.78 | 6.23 | Good |
| C4 | 60.00 | 29.63 | 10.38 | Inferior |
| C5 | 60.00 | 27.03 | 12.97 | Inferior |
| C6 | 60.00 | 23.79 | 16.22 | Poor |
| C7 | 60.00 | 15.00 | 25.00 | Poor |
| 3 | 70.00 | 25.00 | 5.00 | Good |
| C8 | 70.00 | 17.50 | 12.50 | Poor |
| C9 | 80.00 | 7.50 | 12.50 | Poor |
| C10 | 80.00 | 3.40 | 16.60 | Poor |
| 4 | 85.00 | 10.00 | 5.50 | Good |

The “lipid mixture” used in these compositions was a 60:40 blend of glycerol monolaurate and isostearyl alcohol.

The ethanol used in these compositions was absolute alcohol, but had a small amount of fragrance added (1% based on the total formulation).

The propellant used in these compositions was a hydrocarbon blend of propane, butane and isobutane (AP40 from HARP).

The stability of these aerosol compositions was assessed by storing duplicate samples of each at 0° C. for 4 weeks sealed in clear glass aerosol flasks. Instability was manifested by crystallisation of the lipids from the composition, visually assessed through the glass wall of the flasks. Compositions having “good” stability showed no sign of lipid crystallisation in either sample after the 4 weeks. Compositions having “inferior” stability showed lipid crystallisation in one sample after the 4 weeks. Compositions having “poor” stability showed lipid crystallisation in both samples after the 4 weeks.

1. A cosmetic aerosol composition comprising a three-component mixture of propellant, ethanol and a lipid mixture consisting of glycerol monolaurate and isostearyl alcohol at a ratio of from 25:75 to 45:55 by weight, wherein:
(i) the three-component mixture has a propellant content ranging from 40 to 87.5% by weight;
(ii) the three-component mixture has an ethanol content ranging from 10 to 57.5% by weight;
(iii) the three-component mixture has a lipid content ranging from 2.5% by weight up to a maximum percentage equal to the sum of 4.805 and the ethanol content multiplied by 0.115; and
(iv) wherein silicone oil is absent from the composition.

2. The cosmetic aerosol composition according to claim 1, wherein the content of the lipid mixture in the three-component mixture is at least 10% by weight.

3. The cosmetic aerosol composition according to claim 1, wherein the ethanol content of the three-component mixture is from 10 to 45% by weight.

4. The cosmetic aerosol composition according to claim 1, wherein the propellant content of the three-component mixture is from 50 to 87.5% by weight.

5. The cosmetic aerosol composition according to claim 1, wherein the propellant comprises a blend of C3 to C4 hydrocarbons.

6. The cosmetic aerosol composition according to claim 1, wherein the content of the lipid mixture in the total composition, ignoring the propellant therein, is at least 10% by weight.

7. The cosmetic aerosol composition according to claim 6, wherein the content of the lipid mixture in the total composition, ignoring the propellant therein, is at least 15% by weight.

8. (canceled)

9. The cosmetic aerosol composition according to claim 1, comprising a fragrance, the fragrance being considered part of the ethanol in calculating the ethanol content of the composition.

10. (canceled)

11. The cosmetic aerosol composition according to claim 9, wherein the fragrance is present at a level of from 0.5 to 3% by weight.

12. The cosmetic aerosol composition according to claim 1, which is free from aluminium or zirconium antiperspirant salts.

13. The cosmetic aerosol composition according to claim 1, comprising an antimicrobial agent.

14. A cosmetic method of attaining an antiperspirant benefit comprising the topical application of the cosmetic aerosol composition according to claim 1.

15. The cosmetic aerosol composition according to claim 1, wherein the propellant comprises propane, butane, isobutane, or a mixture thereof.

16. The cosmetic aerosol composition according to claim 1, wherein the content of the ethanol in the total composition, ignoring the propellant therein, is from 10% to 95% by weight.

17. The cosmetic aerosol composition according to claim 1, wherein the content of the ethanol in the total composition, ignoring the propellant therein, is from 15% to 90% by weight.

18. The cosmetic aerosol composition according to claim 1, wherein the content of the lipid mixture in the total composition, ignoring the propellant therein, is from 10 to 40% by weight.

19. The cosmetic aerosol composition according to claim 1, wherein the content of the lipid mixture in the total composition, ignoring the propellant therein, is from 12 to 40% by weight.

20. The cosmetic aerosol composition according to claim 1, wherein the propellant content of the three-component mixture is from 50 to 85% by weight.
